(19) United States
(12) Patent Application Publication
(10) Pub. No.: US 2010/0093017 A1
(43) Pub. Date: Apr. 15, 2010

(54) Metabolically Competent Cell Lines

(75) Inventors: Charles Roland Wolf, Dundee Technopole (GB); Shaohong Ding, Dundee Technopole (GB)

Correspondence Address: MYERS BIGEL SIBLEY & SAJOVEC, PO BOX 37428, RALEIGH, NC, 27627, US

(73) Assignee: CXR Biosciences Limited

(21) Appl. No.: 12/574,962

(22) Filed: Oct. 7, 2009

Related U.S. Application Data
(60) Provisional application No. 61/103,998, filed on Oct. 9, 2008.

(51) Int. Cl. C12Q 1/02; C12N 5/00; C12N 5/071; C12N 15/00

(52) U.S. Cl. 435/29; 435/325; 435/366; 435/370; 435/440

(57) ABSTRACT

The present invention provides cell lines that have been transfected with adenovirus expression vectors so that they express at least one metabolically competent or functional cytochrome P450 enzyme. The invention also includes methods of their use, especially in toxicology screens.

This application claims the benefit of U.S. Provisional Application No. 61/103,998, filed Oct. 9, 2008. The entire content of this application is incorporated by reference herein.

The present invention relates to cultured cell lines that have been transfected with adenoviral expression vectors so that they express one or more functional cytochrome P450 enzymes and optionally a reporter gene, the invention includes inter alia methods of producing the cells, products and methods of their use, especially in toxicology screens.

### BACKGROUND

Cell lines currently used for toxicity testing have limited utility because they lack the ability to metabolise the drug or chemical, which overlooks the generation of a more toxic metabolite. Alternatives to animal experimentation for toxicity in drug development need to provide greater reliability in predicting human toxicity. Use of cell lines for prediction of drug metabolism or toxicity in a human subject is limited by the fact that cell lines currently available have lost differentiated cell functions and do not reproduce the characteristics of organs, such as the liver, where toxic effects are most often seen. In particular, most available human cell lines fail to express the cytochrome P450 enzymes (which determine the metabolism and toxicity of many drugs and chemicals) at levels comparable to those found in intact tissues. The cytochromes P450 (P450s), a family of enzymes catalysing oxidation of a great number of xenobiotic chemicals, are usually absent or expressed at only low levels in cultured cells. Moreover, metabolism of xenobiotics can either increase the toxicity through generation of more toxic metabolites, or abrogate the toxic effects through rapid metabolism of the toxin. Existing cell lines therefore do not replicate the influence of cellular metabolism on the toxic effects of chemicals and cannot be taken to be reliable indicators of compound metabolism and toxicity in vivo. To overcome the limitations of cultured cell lines, P450 metabolism needs to be reintroduced. It will generally be desirable to express several P450s and also in many cases cytochrome P450 reductase (CPR) since P450 enzymes are themselves not metabolically active without appropriate reductase activity being present. Cell lines which could be engineered so that they had the ability to metabolise a drug or chemical would offer immediate improvements to the art.

In general, restoration of P450 metabolism requires expression of P450 and CPR transgenes simultaneously at appropriate levels. Many methods have been developed to introduce functioning transgenes into cells. However, when attempting to restore P450 metabolism by expressing several transgenes, the problem remains of obtaining cells in which all transgenes are expressed simultaneously and at the desired levels.

A number of strategies have been developed to express multiple genes, including internal promoters, fusion proteins and internal ribosomal entry site (IRES). The most commonly used strategy in the construction of two gene vectors is the insertion of an IRES element between the two genes. These two genes are transcribed under the control of a single promoter within the vector. However, a disadvantage of this system is that a gene transcribed upstream of an IRES is expressed strongly whereas a gene placed downstream is expressed at lower levels.

A method of restoring the cellular levels of key metabolic enzymes would therefore be valuable in improving the ability of cultured cell lines to predict the in vivo metabolism and toxicity of applied chemicals.

The present invention provides a novel method of restoring the functions of xenobiotic metabolism in cultured cell lines by using an adenoviral-based multiple P450 expression system. This invention advantageously allows the expression of human P450s of choice in a wide variety of mammalian cell lines, thereby replicating any chosen profile of P450-mediated metabolism and providing in vitro prediction of compound metabolism and metabolically activated toxicity.

### BRIEF SUMMARY OF THE DISCLOSURE

According to a first aspect of the invention there is provided a cell derived from a cultured cell line that expresses one or more metabolically active cytochrome P450s, the cell containing an adenovirus expression vector that comprises nucleic acid sequences encoding one or more different cytochrome P450s, the nucleic acid sequences encoding the one or more different cytochrome P450s being positioned in tandem and separated from one another by self-processing cleavage sequences.

This present invention provides cell lines capable of predicting toxicity of drugs or other chemicals in humans that could be used as a replacement for animals in safety testing. Our approach has been to generate transgenic cell lines that provide good prediction of human toxicity by introducing expression of multiple human P450s into cells carrying “reporter” genes. The reporter genes are artificial transgenes designed to signal early stages of various types of toxicity such as oxidative stress, hypoxia, DNA damage, onset of programmed cell death (apoptosis), inflammation or abnormal cell division. Their ability to reliably predict toxicity of an applied compound often depends on appropriate metabolism of the compound which is assured by the presence of the P450s.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Preferably, the cell derived from the cell line is transfected with the adenovirus expression vector. Preferably, the cell is stably transfected with the vector.

Preferably the cell line is a mammalian cell line and more preferably is a human cell line.

A “cell line” is cells grown in tissue culture and representing generations of a primary culture. A cell line is a permanently established cell culture that will proliferate indefinitely given appropriate fresh medium and space, cell lines are distinct families of cell types grown in culture and cells in the same line are typically clones. Different cell lines have different features which are useful in molecular biological applications, examples of cell lines that can be used in the present application include but are not limited to the ARE, CHO, MCF-7, HeLa, A2780, HepG2 cell lines.

Preferably, the cell is a cell that in vivo is associated with having an endogenous P450 function. For example the cell maybe derived from a kidney, brain, lung, heart, skin, liver or placental cell line. More preferably the cell is from a hepatic cell line. In other embodiments of the invention the cell line may be a tumour cell line or maybe a cell line derived from a tissue that is not associated with P450 metabolism.

The NADPH-dependent cytochrome P450 reductase (CPR) is a membrane bound protein localized in the ER membrane. CPR donates electrons from the two-electron donor NADPH to the heme of P450. A functional requirement for cytochrome P450s to be expressed is that it receives electrons from CPR. Accordingly, in some embodiments of the invention where the cells from the cultured cell line do not have an inherent electron donating CPR capacity the adenoviral expression vector further includes a nucleic acid sequence encoding CPR, the CPR being positioned in tandem with the nucleic acid sequences encoding the one or more different cytochrome P450s and separated therefrom by further self-processing cleavage sequence. Thus preferably, the cell derived from the cell line also expresses a P450 reductase that is either inherent in the cell or is included and added to the adenoviral expression vector. In this way, by either the cell having an inherent CPR or by having one manufactured thereinto, the cell is capable of expressing functional P450 with a metabolic capability. It will be appreciated that some cell lines that already have CPR will be transfected with adenovirus expression vectors that do not have a CPR and associated self processing sequence whilst cell lines deficient of CPR will be transfected with adenovirus expression vectors that do contain a CPR and associated self processing sequence. Accordingly, the transfected expression vector is selected according not only to the cell line but also the type and number of functional P450s which it is desired to express.

Preferably, the cell derived from a cell line expresses multiple P450s. In embodiments of the invention the cell expresses 2, 3, 4, 5, 6, 7, 8 or more P450s.

Cytochrome P450 includes the CYP1 family (CYP1A1; CYP1A2; CYP1B1), CYP2 family (CYP2A6; CYP2A13; CYP2B6; CYP2C8; CYP2C9; CYP2C19; CYP2D6; CYP2E1; CYP2F1; CYP2J2; CYP2R1; CYP2S1; CYP2W1), CYP3 family (CYP3A4; CYP3A5; CYP3A7; CYP3A43), CYP4 family (CYP4A11; CYP4A22; CYP4B1; CYP4F2) and CYP>4families (CYP5A1, CYP8A1, CYP19A1, CYP21A2, CYP26A1). The P450S that can be expressed by the cells of the following invention include any one or more of the aforementioned P450s.

Preferably, the P450s are human P450s. Representative P450 include but are not limited to CYP2D6, CYP2E1, CYP1B1 and CYP3A4.

Preferably, the expression of each of the P450s is driven by a self-processing cleavage sequence. Thus in embodiments of the invention in which the cell expresses 2, 3, 4, 5, 6, 7, 8 or more P450s the number of self-processing cleavage sequences will be commensurate as each P450 has its own dedicated self-processing cleavage sequence.

Preferably, the adenovirus expression vector further includes at least one reporter sequence or transgene and an associated self-processing cleavage sequence.

Preferably, the cell derived from the cell line co-expresses the reporter transgene that is responsive to drug or chemical induced toxicity. For example and without limitation, the reporter transgene comprises regulatory sequences responsive to oxidative stress (haemoxygenase 1 promoter); antioxidant response (ARE); inflammation (NF-kB); cell cycle advance (AP-1); DNA damage (p53); apoptosis (p21/Waf1); hypoxia (HRE) and other cell stress responsive sequences (XRE, Hsp70, GRE). The readout from these reporter genes are either luciferase or CXR's proprietary epitope-tagged β-hCG.

A “self-processing cleavage site” or “self-processing cleavage sequence” is defined herein as a post-translational or co-translational processing cleavage site sequence. Such a “self-processing cleavage” site or sequence refers to a DNA or amino acid sequence, exemplified herein by a 2A site, sequence or domain or a 2A-like site, sequence or domain. As used herein, a “self-processing peptide” is defined herein as the peptide expression product of the DNA sequence that encodes a self-processing cleavage site or sequence, which upon translation, mediates rapid intramolecular (cis) cleavage of a protein or polypeptide comprising the self-processing cleavage site to yield discrete mature protein or polypeptide products.

Preferably, the self-processing cleavage sequence is a 2A sequence and is derived from a mammalian virus selected from the group comprising foot and mouth disease virus (FMDV), cardiovirus encephalomyocarditis virus (EMCV), Theiler's murine encephalitis virus (TMEV), equine rhinitis A virus (ERAV), equine rhinitis B virus (ERAV) and porcine teschovirus-1 (PTV-1; formerly porcine enterovirus-1).

Alternatively, the 2A sequence is derived from an insect virus selected from the group comprising Thoseaasigna virus (TaV), infectious flacherie virus (IFV), Drosophila C virus (DCV), acute bee paralysis virus (ABPV) and cricket paralysis virus (CrPV).

The adenovirus vectors carry DNA coding sequences for the P450s of choice and optionally P450 reductase if required in tandem, each component being separated by a dedicated 2A sequence. In some embodiments the adenovirus vectors further include a reporter transgene and dedicated 2A sequence so that the reporter transgene can be co-expressed with the P450s.

The major challenge addressed by the present invention has been to achieve co-expression of multiple P450s in cultured cells. Previously, this might have required many time-consuming transfection and cloning operations. In the present invention an innovative strategy has been developed to allow expression of multiple P450s in almost any cell line of interest. This exploits the availability of adenovirus vectors for transient expression of transgenes in cell lines and the properties of the 2A peptide sequence coded by the Foot and Mouth Disease Virus which causes a break in peptide chains produced during gene translation. Combined, these two features allow the simultaneous expression of multiple proteins from a single viral gene transfer.

According to a further aspect of the invention there is provided a cell from a cultured cell line that expresses one or more metabolically active cytochrome P450s, the cell comprising an adenovirus expression vector and one or more nucleic acid sequences encoding cytochrome P450s selected from the CYP1, CYP2, CYP3, CYP4 and CYP>4 families each selected cytochrome P450 being positioned in tandem with interposed 2A self processing sequences separating them and wherein the cell has a CPR function that is either inherent to the cell line or is provided by a nucleic acid sequence encoding CPR and 2A self-processing sequence.

Preferably, the cell is a human hepatocyte and preferably the expressed functional P450s are human P450s. In this way human metabolism in human cells in vitro may be assessed.

According to a yet further aspect of the invention there is provided a method of producing the cells of the first aspect of the invention, the method comprising stably transfecting a cell derived from a cultured cell line with an adenovirus expression vector that comprises nucleic acid sequences encoding one or more different cytochrome P450s, the nucleic acid sequences encoding the one or more different cytochrome P450s being positioned in tandem and separated from one another by self-processing cleavage sequences, the vector optionally further including a nucleic acid sequence encoding CPR, the CPR being positioned in tandem with the nucleic acid sequences encoding the one or more different cytochrome P450s and separated therefrom by further self-processing cleavage sequence.

According to a yet further aspect of the invention there is provided use of the cells derived from a cell line as herein before described, as models for drug metabolism and/or for screening candidate compounds for toxic effects via metabolic activation. A drug or candidate compound can be any compound, agent, or molecule that is known to have or may have a therapeutic, diagnostic or other use when administered to an animal, e.g., a human.

According to a yet further aspect of the invention there is provided a method of assessing human P450 metabolism of a candidate therapeutic or other compound in vitro in a cell, comprising exposing the cell of the first aspect of the invention to the candidate therapeutic or other compound and measuring metabolite production.

According to a yet further aspect of the invention there is provided a method of assessing potential toxicity of a candidate therapeutic or other compound in vitro as a result of human P450 metabolism of the candidate therapeutic, comprising exposing the cell of the first aspect of the invention to the candidate therapeutic or other compound and measuring cytotoxic effects.

In the embodiment of the invention where the adenovirus expression vector further includes a reporter transgene the expression of the transgene products may be used as an indicator of the metabolic status and/or cytotoxicity.

It will be appreciated that the methods of the present invention advantageously improve the relevance of in vitro studies of screens to human metabolism.

Preferred features ascribed to each and every aspect of the invention apply mutatis mutandis to each and every aspect of the invention.

### BRIEF DESCRIPTION OF THE DRAWINGS

FIG. 1 shows the strategy for construction of bicistronic adenoviral vectors. FIG. 1A shows the modification of FMDV 2A (F2A) sequence (SEQ ID NOS: 1 and 2) and FIG. 1B shows the bicistronic construct.

FIG. 2 shows constructs used to generate recombinant adenoviruses which were then infected into the developed reporter cells individually or in a group of different P450s to express various combinations of P450s and reporters in cultured cells.

FIG. 3 shows expression of multiple P450s and P450 reductase in CHO cells by immunoblotting. Each lane was loaded with 30 μg total cell lysate protein from cells infected with: (1) adenovirus only; (2) CYP2D6(His)CPR fusion vector; (3) CYP2A6.F2A.CPR vector; (4) CYP2A6.F2A.CYP1A1; (5) CYP3A4. F2A.CPR; (6) CYP3A4.F2A.CPR+CYP2D6(His)CPR fusion; (7) CYP3A4.F2A.CPR+CYP2A6. F2A.CYP 1 A1; (8) CYP3A4.F2A.CPR+CYP2A6.F2A.CYP1A1+CYP2D6(His) CPR fusion; (9) 10 μg mouse liver microsomal protein

FIG. 4 shows ARE induction by viral transduction and BaP.

FIG. 5 shows ARE induction in MCF-7/ARE cells infected with P450s.

FIG. 6 shows adenovirus-mediated co-expression of human CYP3A4 and CPR in CHO cells.

FIG. 7 shows human CYP3A4 activity in transduced CHO cells. Adenovirus was transduced into CHO cells and enzyme activity was measured 2 days after transduction. Activity is shown in pmol/min/10⁶ cells.

FIG. 8 shows induction of ARE by viral transduction and antioxidants.

FIG. 9 shows cytotoxicity of acetaminophen (APAP) and tamoxifen in human hepatocytes expressing CYP3A4.

FIG. 10 shows ATP assay for acetaminophen (APAP) in viral-transduced HepG2 cells.

FIG. 11 shows ATP assay for tamoxifen in viral-transduced HepG2 cells.

### DETAILED DESCRIPTION

### Chemicals and Cell Culture

Unless otherwise stated, all chemicals and all media supplements for cell culture were purchased from Sigma-Aldrich Co. Ltd. (Dorset, United Kingdom). HepG2 (human hepatoblastoma), MCF7 (human breast carcinoma), and Chinese hamster ovarian carcinoma (CHO) cell lines were purchased from ECACC. The growth medium for MCF7 and HepG2 cells was DMEM supplemented with 10% fetal bovine serum and antibiotics. The growth medium for CHO cells was RPM-1640. All cells were cultured at 37′C in 5% CO₂, and passaged every 3 to 4 days.

### Bicistronic Expression Constructs

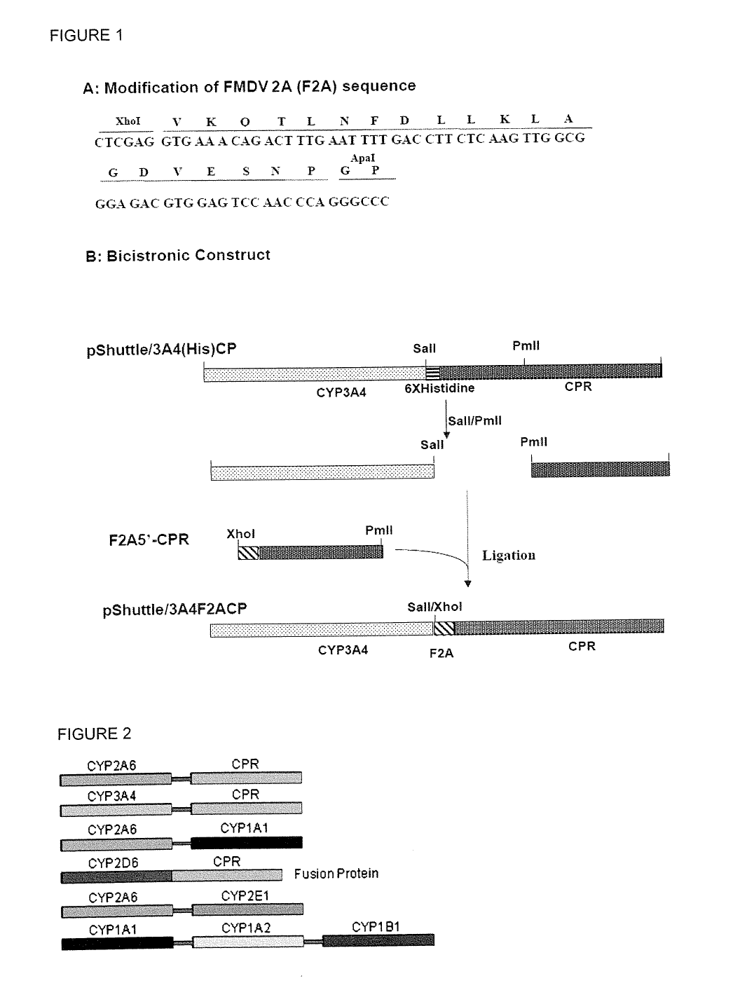

Plasmid pET32hCGZsGreen Final is already available and contains a cassette of hCG-F2A-ZsGreen. This cassette was cut off from plasmid pET32hCGZsGreen Final by using restriction enzymes KpnI/PmeI and generated 2 kb fragment was inserted into KpnI/EcoRV double digested vector pcDNA3 to construct plasmid pcDNA/hCG-ZsGreen. The F2A sequence was cut off from plasmid pcDNA/hCG-ZsGreen by BamHI/EcoRI digestions and the generated 0.37 kb fragment was cloned into BamHI/EcoRI sites in plasmid pUC18. The F2A was modified by site directed mutagenesis to remove an internal ApaI site 60 base pairs upstream 5′-end of F2A sequence with oligo pair P56F/R, and then a XhoI site was introduced upstream of first codon of F2A (oligos P53F/R). This plasmid was named as pUC18/F2A, and was ready to be cut off for cloning (FIG. 1A). CPR cDNA was cut off from plasmid pcDNA/HR with enzymes KpnI/XbaI, the 2 kb fragment was cloned into the KpnI/XbaI sites of pUC18. Then the 5′-half of CPR (from ATG start codon to 50 base pairs downstream the PmII site) was amplified by PCR with primers P52F/P52R. The primer P52F contains an ApaI site upstream of ATG codon of CPR and the ApaI site was introduced into the predicted product (1.0 kb) of PCR. After being separated by electrophoresis on an agarose gel, the DNA fragment was extracted from the agarose gel, and then cloned into vector pCR2.1-TOPO by using TOPO TA Cloning kit (Invitrogen Corp. Cat. no. K4575-01) to generate a plasmid pCR2.1/5′-half CPR. The 1.0 kb fragment of 5′-half CPR was cut off from the plasmid pCR2.1/5′-half CPR by ApaI/HindIII and inserted into the ApaI/HindIII sites of pUC/F2A to generate the plasmid pUC18/F2A-5′-Half CPR (FIG. 1B).

The CYP1A1 and CYP2A6 cDNAs used were obtained as Image Clones 5123393 and 40006068 respectively and were sequenced to confirm no mutation in the sequence.

Adenovirus Production. The recombinant adenoviral constructs containing bicistronic P450 coding sequences or containing a fused P450-CPR and a control pShuttle/CMV were linearized with PmeI and were transformed into BJ5183-AD-1 cells. The recombinant adenoviral constructs were identified by PacI digestion. Adenoviruses were produced by transfection of PacI digested adenoviral constructs into AD-293 cells. The recombinant adenovirus and the control virus Ad-mock were amplified in Ad293 cells to generate stocks of adenovirus according to the Manufacture's protocols (ViralPower Adenoviral Expression System, Invitrogen). The titer of each viral stock was determined by plaque assay (AdEasy XL Adenoviral Vector System, Instruction Manual, Stratagene) or immunology assay (AdEasy Viral Titer Kit, Instruction Manual, Stratagene). Titers of the stocks were at the range of 1×10⁸ to 1×10⁹ plaque-forming units (pfu)/ml or infection units (ifu)/ml.

### Adenoviral Transduction and Effects on Luciferase Reporter Activity in ARE32 Cells

ARE32 cells were seeded in 24-well plates at 2×10⁵ cells per well and cultured overnight. Cells were transduced for 2 h with up to three Adeno-P450s at MOI value of 27 pfu/cell in 0.5 ml of culture medium, after which the medium was removed and replaced with fresh medium. Cells were incubated for an additional 48 h and then were treated with chemicals in serum-free medium for 1 day. Then cells were harvested and lysed. The Luciferase Reporter Assay System (Promega) was used to examine reporter gene activity in cell lysates.

### CYP3A4 and CYP2D6 Activity Assays—Midazolam and Bufuralol Hydroxylation

Samples were analyzed by LC-MS/MS for 1-OH-bufuralol and 1-OH-midazolam using a CTC PAL autosampler, an Agilent 1100 pump and a PE Sciex API 3000 mass spectrometer. An electro spray was used as the ionization source. An Agilent Zorbax SB-C18 column (2.1×50 mm, 5 μm) was used for the separation. The chromatography was performed with a mobile phase A (CH₃OH:1 M CH₃COONH₄:HCOOH:H₂O, 50:2:0.755:950 v/v/v/v) and mobile phase B (CH₃OH:1 M CH₃COONH₄:HCOOH:H₂O, 900:2:0.755:100 v/v/v/v) using a linear gradient from 0 to 100% B in 3 min, at 100% B until 3.1 min and from 3.1-3.5 min back to 100% A with a flow-rate of 0.3 ml/min. The column temperature was maintained at 60° C. LOQs were determined relative to baseline noise (S/N=10).

### Cytochrome P450 Reductase Activity Assay—Cytochrome C Reduction

Activity of CPR was assayed under aerobic conditions at 37° C. in 1 mL incubation mixtures containing 0.3 M potassium phosphate (pH 7.7), 50 μM cytochrome c, and total cellular protein (10 μg). Reactions were initiated by the addition of 10 μL 5 mM NADPH, and the rate of cytochrome c reduction was determined spectrophotometrically at 550 nm based on extinction coefficient for cytochrome C ε=21.4 mM cm⁻¹. The rate of the enzyme-catalyzed reaction was determined by subtracting the rate of the reaction occurring in the absence of protein. Product formation was linear with respect to protein concentration and incubation time.

### CYP2A6 Activity Assay—Coumarin 7-Hydroxylation

Coumarin (50 μM) was added into the adenoviral transducted HepG2 cells in 24-well plate with 4×10⁵ cells in 0.5 ml medium and incubated with cells for 4 hrs. Then 25 μL of the medium were mixed with 75 μL of H₂O and 5 μl of 1M Tris (pH 9.0). The fluorescence was measured in 96-well plates by using ELISA reader (Fusion, Packard) with excitation at 365 nm and emission at 460 nm (bandwidth of 40 nm). All assays were performed in duplicate, and eight concentrations of 7-Hydroxycoumarin (0 to 100 pmol/well) were included to construct a standard curve. Activity of CYP2A6 is expressed as picomoles of 7-Hydroxycoumarin formed per minute, per 10⁶ cells or mg protein (pmol/min/10⁶ cells or pmol/min/mg protein).

### CYP1A1 Activity Assay—Ethoxyresorufin Deethoxylation

Ethoxyresorufin (5 μM) and salicylamide (3 mM) were added into the adenoviral transducted HepG2 cells in 24-well plate with 4×10⁵ cells in 0.5 ml medium and incubated with cells for 4 hrs. Then 25 μL of the medium were mixed with 75 μL of H₂O. The fluorescence (in 100 μL) was measured by using ELISA reader (Fusion, Packard), with excitation at 530 nm and emission at 590 nm. All assays were performed in duplicate, and seven concentrations of resorufin (0 to 32 pmol/well) were included to construct a standard curve. Activity of CYP1A1 is expressed as picomoles of resorufin formed per minute, per 10⁶ cells or mg protein (pmol/min/10⁶ cells or pmol/min/mg protein).

### Immuno Blot Assay of CPR and CYP Protein Expression

Polyclonal antibody raised in sheep against human CYP3A4 (NF14) and CYP2D6; rabbit anti-CPR were obtained from Biomedical Research Centre, Dundee University, UK. Polyclonal antibody raised in rabbit against human CYP1A1 and CYP2A6 were from CXR Biosciences Ltd. Dundee, UK. Horseradish peroxidase (HRP) conjugated ECL anti-rabbit and anti-sheep antibodies were purchased from GE Healthcare, UK limited (Little Chalfont Buckinghamshire, UK). For the whole-cell extracts, cells were harvested by cell lifter and lysed in lysis buffer containing 10 mM sodium phosphate (pH 8.0), MgCl₂ (2 mM), EDTA (1 mM) and dithiothreitol (2 mM). Cells were lysed by sonication using an MSE Soniprep (two 5 second bursts at amplitude microns of 12 with sample kept on ice). Protein concentrations were determined using a commercially available protein assay kit (DC Protein Assay, Bio-Rad). Total cellular proteins were separated on a 10% SDS-polyacrylamide gel and electroblotted onto nitrocellulose membranes and probed with primary antibody. Antibody binding was visualized on X-ray film by enhanced chemiluminescence using the ECL kit from Amersham Pharmacia Biotech.

Recombinant adenoviral vectors containing FMDA 2A peptide conferring efficient bicistronic gene expression in cultured cells were generated. FIG. 1 shows an example of the structure of an expression construct and FIG. 2 shows a variety of constructs utilised for multiple P450s and P450 reductase expression.

### EXAMPLE 1

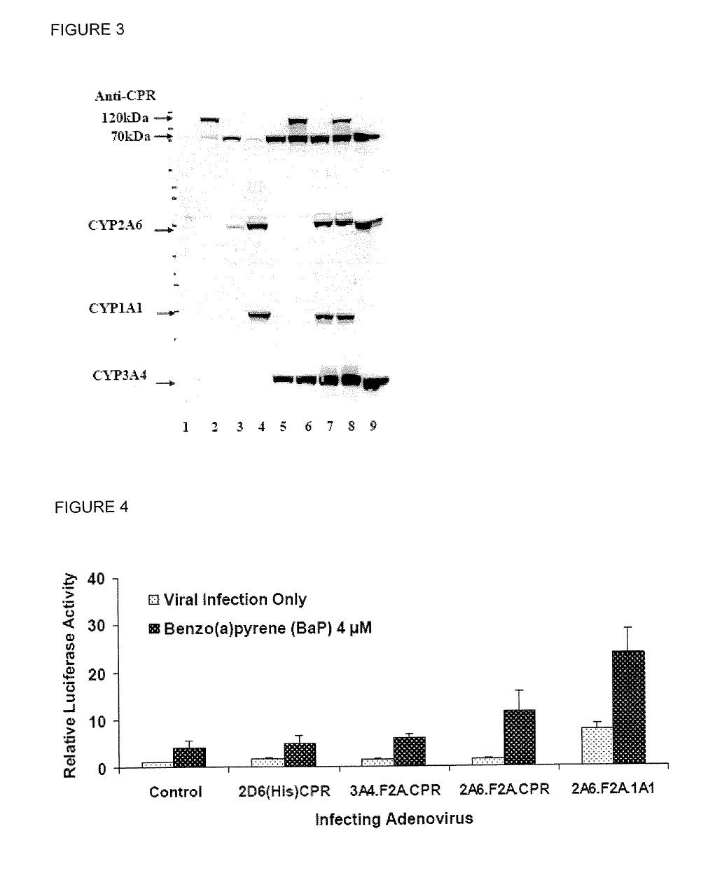

Next we explored the possibility of co-expression of multiple P450s and CPR by co-transduction with the multiple recombinant adenoviruses. Adenovirus vectors were generated carrying DNA coding sequences for the P450s of choice and cytochrome P450 reductase (CPR) in tandem, separated by 2A sequences. Altogether, six recombinant adenoviruses were generated expressing CPR and different P450s with up to three P450 genes in one adenovirus. An adenovirus containing CYP2A6 and CYP1A1 (Ad2A6.F2A.1A1) and an adenovirus containing a fused gene of CYP2D6 and CPR (Ad2D6(His)CPR) were generated. Three recombinant adenoviruses at total MOI value of 27 were used to transduce CHO cells and successfully achieved co-expression of up to four P450s (CYP3A4, CYP2D6, CYP2A6, & CYP1A1) together with CPR (FIG. 3). By infecting cell lines with multiple adenoviruses, we were able to achieve high levels of simultaneous expression of up to four P450s (CYP3A4, CYP2D6, CYP2A6 and CYP1A1) (FIG. 3). P450 transgene function was confirmed by immunoblot analysis of cell lysates. Immunoreactive bands corresponding to CPR and each of the P450s demonstrated that adenovirus infection did indeed result in expression of each of the desired proteins. Results therefore confirmed co-expression of multiple P450s and CPR in cells.

### EXAMPLE 2

In cell line ARE32, the antioxidant responsive element (ARE), activated by Nrf2, is used to drive a luciferase gene as reporter. A functional ARE is found in the 5′ flanking region of genes encoding NQO1, multiple GST isozymes and many other anticarcinogenic/antioxidant genes. Induction of these genes confers cytoprotection against carcinogenesis and acts to minimize the effects of the toxic insult. Therefore, measurement of ARE induction provides useful information into the particular mechanism of toxicity. We tested whether multiple P450s and P450 reductase were capable of expression in ARE32 cells. Table 1 shows the levels of P450 activity obtained in transduced ARE32 cells, indicating that up to four P450s and CPR were introduced and expressed in cells. ARE32 control values are from ARE32 cells without virus infection. ARE32 Transduced values are from ARE32 cells infected with three viruses: Ad3A4.F2A.CPR; Ad2D6(His)CPR; and Ad2A6.F2A.1A1 at a total MOI value of 27.

TABLE 1

| | ARE32 | ARE32 |
|---|---|---|
| Enzyme activity | Control | Transduced |
| CPR: cytochrome c reduction (nmol/min · | 23.1 | 79.1 |
| mg protein) | | |
| CYP3A4: 1′-hydroxymidazolam formation | 0.1 | 4.4 |
| (pmol/min/mg protein) | | |
| CYP2D6: 1′-hydroxybufuralol formation | 0 | 16.2 |
| (pmol/min/mg protein) | | |
| CYP2A6: 7′-hydroxycoumarin formation | 0 | 58.2 ± 1.5 |
| (pmol/min/10⁶ cells) | | |
| CYP1A1: Resorufin formation (pmol/min/10⁶ | 0 | 73.7 ± 0.9 |
| cells) | | |

Benzo(a)pyrene (BaP), a carcinogen found in coal tar, diesel exhaust fumes and charred food. It is toxic after metabolic activation by CYP1A1. We tested the effects of transduced CYP expression on ARE induction following application of BaP. P450s were introduced into the ARE reporter cells as indicated in FIG. 4, and then the cells were exposed to BaP (4 μM). Results showed that BaP treatment provoked a strong induction of ARE (>20-fold) in cells that express both human CYP1A1 and CYP2A6 cDNAs, but showed no induction in cells expressing CYP3A4 and CYP2D6.

Infection of MCF-7 human breast cancer cells with the CYP3A4-CPR and CYP2A6-CYP1A1 vectors and the CYP2D6/CPR fusion vector and incubation with appropriate P450 substrates in the culture medium for one hour resulted in substantial rates of metabolism of the substrate compounds compared to little or no metabolism in uninfected MCF-7 cells. We believe that this is the first time that cell lines simultaneously expressing high levels of multiple human P450s has been achieved. Accordingly results show that a model has been generated for testing chemical metabolism and the toxic effects of inter-mediated metabolites simultaneously.

### EXAMPLE 3

Toxic responses are complex, but in their early stages are often associated with increased expression of ‘stress induced’ genes. Artificial reporter genes whose expression is under the control of regulatory DNA elements associated with such ‘stress induced’ genes can therefore be used as ‘engineered biomarkers’ of developing toxic responses. Reporter genes can be designed to act as biomarkers of a variety of cellular stress responses associated with early stages of toxicity. These include regulatory sequences responsive to oxidative stress (haemoxygenase 1 promoter); antioxidant response (ARE); inflammation (NF-kB); cell cycle advance (AP-1); DNA damage (p53); apoptosis (p21/Waf1); hypoxia (HRE) and other cell stress responsive sequences (XRE, Hsp70, GRE). The readout from these reporter genes are either luciferase or CXR's proprietary epitope-tagged β-hCG.

P450-mediated metabolism of chemicals can either increase or decrease their toxicity through generation of more toxic metabolites or metabolism of the toxic compounds respectively. To establish the importance of co-expressed P450s in determining toxicity reporter responses, we compared the effects of various compounds on reporter gene expression with various co-expressed P450s.

First, we examined the effects of benzo(a)pyrene (BaP), a highly toxic carcinogen, on expression of a reporter gene consisting of the antioxidant response element (ARE) driving expression of a luciferase readout gene in MCF-7 cells. BaP provoked a more than 20-fold induction of the reporter gene in cells expressing both human CYP1A1 and CYP2A6, but showed no induction in cells expressing CYP3A4 or CYP2D6.

In further experiments, we examined induction of the ARE reporter in MCF-7 cells expressing CYP3A4 and CPR, CYP2A6 and CPR, CYP2D6 and CPR or CYP2A6 and CYP1A1 by either butylated hydroxyanisole, an antioxidant widely used as a food preservative (BHA-20 μM) or 7-ethoxycoumarin, an antioxidant (7-E-100 μM). We found that treatment with either compound provoked a more than 32-fold induction of the reporter in cells that express human CYP1A1 and CYP2A6, but that little or no induction was present in control cells or in the presence of CYP3A4 or CYP2D6.

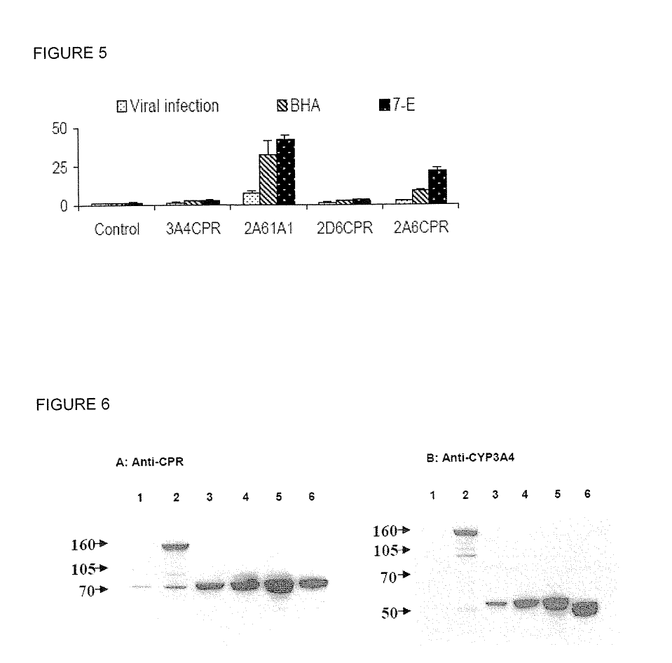

With reference to FIG. 5, expression of the luciferase reporter readout is evaluated in comparison to control MCF-7/ARE cells not expressing P450 transgenes (first bar=1).

### EXAMPLE 4

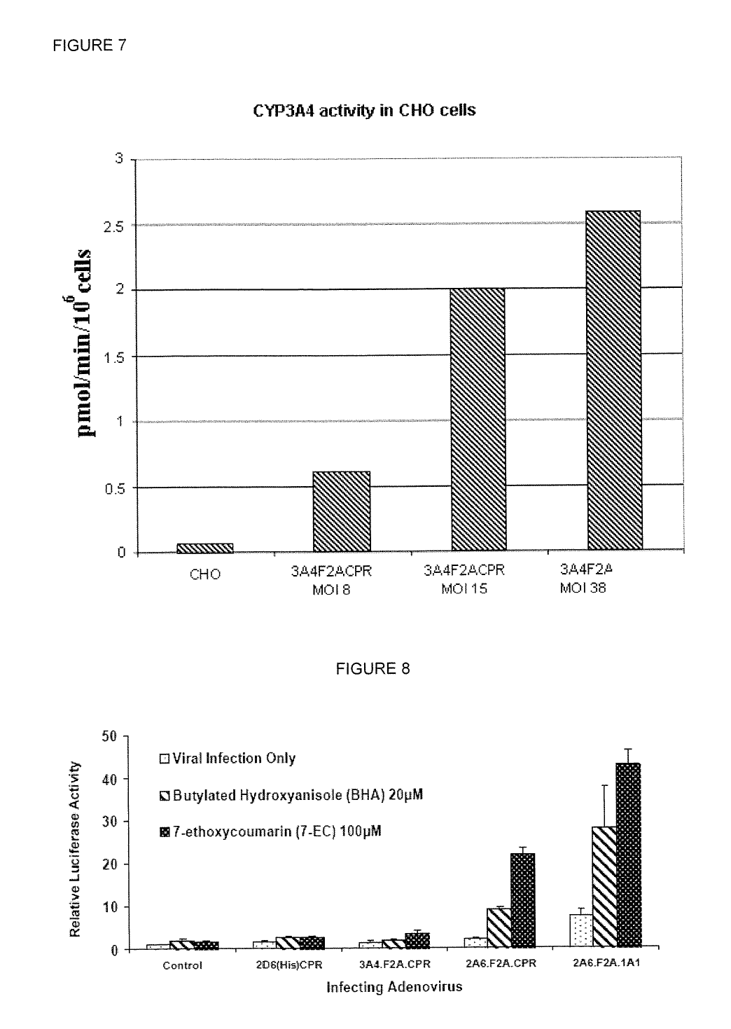

In order to achieve a high level of P450 activity the appropriate amount of adenoviruses used for transducing cells should be optimised to give ˜100% of transduction. However, the amount of adenovirus cell surface receptors varies greatly among different cell types. If too much virus is used, it will cause cytotoxicity or other undesired effects in cells. Therefore, we first tested the optimal multiplicity of infection (MOI) value in CHO cells. In the experiment shown in FIG. 6, CHO cells were transduced with Ad3A4.F2A.CPR at MOI values of 8, 15 and 38 pfu/cell and adenovirus Ad3A4(His)CPR, which generates a fused CYP3A4(His)CPR protein (˜120 Kda), used as a control. After additional 60 h incubation, the level of P450 protein in transduced cells were determined by Western blot analysis and results showed that fusion adenovirus produced a fused CYP3A4(His)CPR protein of ˜120 KDa and the bicistronic construct (Ad3A4.F2A.CPR) produced two processing products corresponding to the individual ‘cleaved’ proteins CYP3A4-F2A and CPR. There was no uncleaved CYP3A4-2A-CPR protein found in the cell lysate. 1′-hydroxylation of midazolam in transduced CHO cells was also measured (FIG. 7). Results indicated that addition of the 2A peptide to CYP3A4 protein did not affect the function of CYP3A4 and enzyme activity of CYP3A4 was gradually elevated with the increase of MOI value. Results confirm that FMDV 2A peptide confers efficient bicistronic gene expression and cleavage in cultured cells.

### EXAMPLE 5

Butylated hydroxyanisole (BHA) is an antioxidant used as a food additive (E320). We tested whether BHA and 7-ethoxycoumarin were capable of inducing ARE reporter activity in these cells because it is necessary for them to be metabolized by P450s into compounds that induce ARE-driven gene promoters. For example, BHA is O-demethylated by cytochrome P450 to yield tert-butylhydroquinone which is a more potent inducer of ARE than BHA. We examined induction of the ARE reporter in ARE32 cells expressing CYP3A4 and CPR, CYP2A6 and CPR, CYP2D6 and CPR or CYP2A6 and CYP1A1 by either butylated hydroxyanisole (BHA) at 20 μM or 7-ethoxycoumarin (7-EC) at 100 μM (FIG. 8). Significant ARE reporter induction was seen with CYP2A6 but not with CYP3A4 or CYP2D6. Greatest induction (>35-fold) was seen when CYP2A6 was co-expressed with CYP1A1. Results show that induction of ARE by P450-dependent metabolites of butylated hydroxyanisole & 7-ethoxycoumarin.

### EXAMPLE 6

This study aimed to evaluate the adenovirus-mediated expression of CYP3A4 and to test the toxicity of two compounds (acetaminophen and tamoxifen) and their CYP3A4-dependent metabolites in HepG2 cells. CYP3A4 and P450 reductase were delivered into HepG2 cells (Hep-3A4) by adenoviral transduction at Multiplicity of Infection (MOI)=8. Data confirmed that CYP3A4 was active. The adenovirus Ad-mock was used to transduce HepG2 cells at MOI=8 as control (Hep-mock). CYP3A4 activity was determined and compared in Hep-3A4 cells and cryopreserved human hepatocytes. The level of activity (1′-Hydroxylation of midazolam) in Hep-3A4 cells was ˜30-40% of those in cryopreserved human hepatocytes. The level of activity observed in Hep-3A4 cells compared to human hepatocytes is within the normal population range.

TABLE 2
P450 Activities in Hep-3A4, Human Hepatocytes and HepG2 cells

| | CYP3A4 Activity | |
|---|---|---|
| Cells | 1′-Hydroxymidazolam | 4′-Hydroxymidazolam |
| HepG2 | <Detection Limit | <Detection Limit |
| Hep-3A4 | 1.99 ± 0.23 | 0.37 ± 0.05 |
| (MOI = 8) | pmol/min/mg protein | pmol/min/mg protein |
| | 1.45 ± 0.06 | 0.27 ± 0.02 |
| | pmol/min/10⁶ cells | pmol/min/10⁶ cells |
| Cryopreserved Human | 4.59 ± 0.55 | 0.36 ± 0.06 |
| Hepatocytes | pmol/min/mg protein | pmol/min/mg protein |
| | 4.64 ± 0.08 | 0.37 ± 0.02 |
| | pmol/min/10⁶ cells | pmol/min/10⁶ cells |

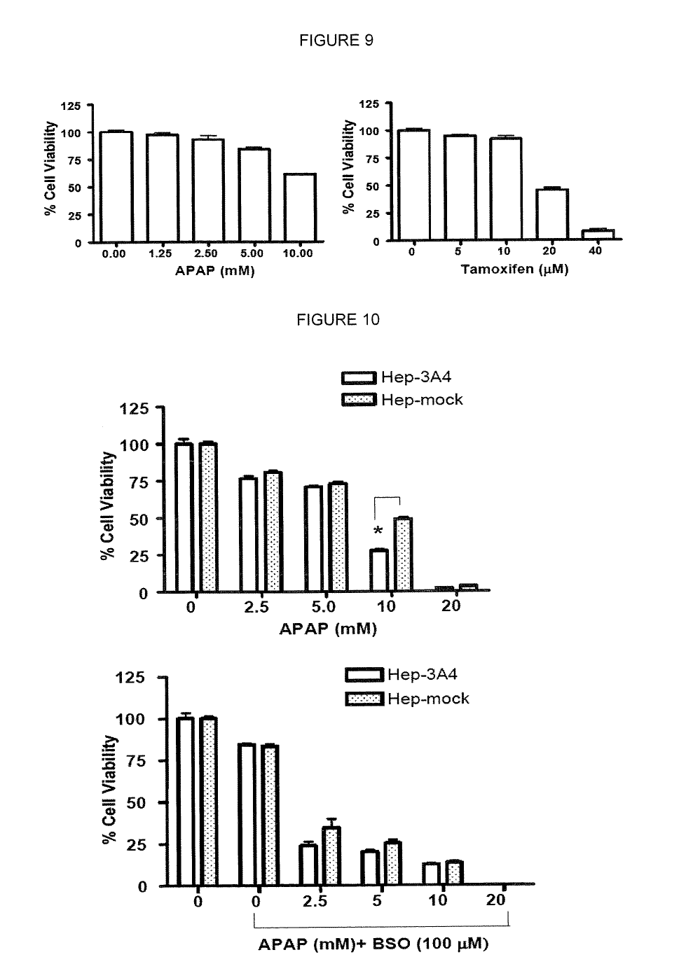

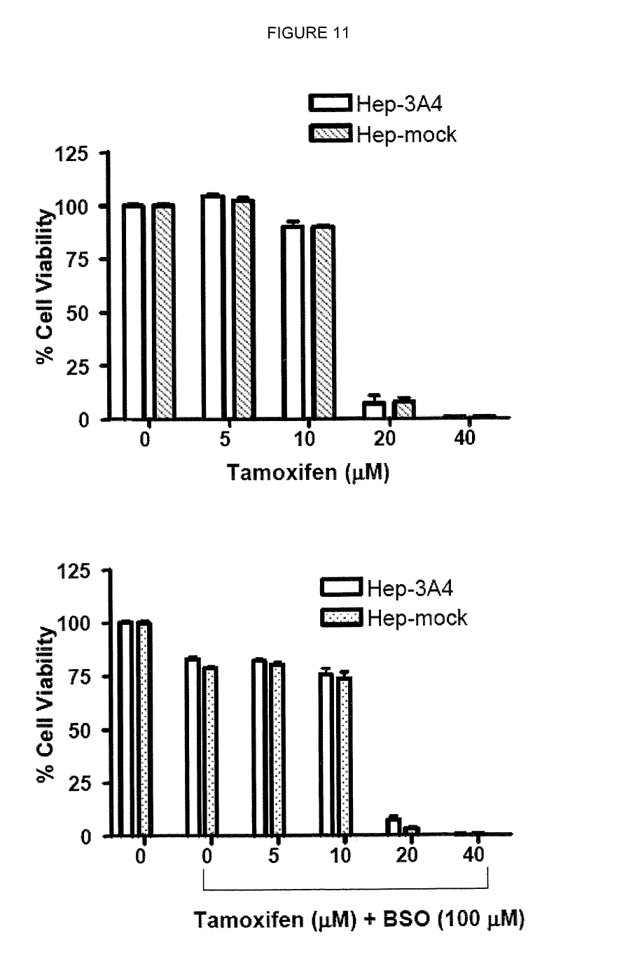

The cytotoxicities of acetaminophen and tamoxifen were determined by the ATP depletion assay. A dose-dependent decrease in cell viability was observed following treatment with the Test Items in adenoviral transduced HepG2 cells (Hep-3A4 and Hep-mock) and cryopreserved human hepatocytes (FIG. 9). CYP3A4-related toxic activation of acetaminophen was observed at concentration of 10 mM (FIG. 10). Depletion of glutathione by simultaneously exposing the cells to 100 μM of BSO resulted in a significant sensitisation of both Hep-3A4 and Hep-mock cells to acetaminophen, shifting the concentration-response to the left such that there was no apparent additional CYP3A4 related cytotoxic effect observed at the concentrations used. P450-related toxic activation of tamoxifen was not observed in this study (FIG. 11). Depletion of glutathione by pre-treatment with BSO had no effect on the cytotoxicity of tamoxifen in either Hep-3A4 or Hep-mock cells. This suggests that tamoxifen cytotoxicity is not P450-dependent.

1. A cell from a cultured cell line that expresses one or more metabolically active cytochrome P450s, the cell containing an adenovirus expression vector that comprises nucleic acid sequences encoding one or more different cytochrome P450s, the nucleic acid sequences encoding the one or more different cytochrome P450s being positioned in tandem and separated from one another by self-processing cleavage sequences.

2. The cell according to claim 1 wherein it is stably transfected with the vector.

3. The cell according to claim 1 that is from a mammalian cell line.

4. The cell according to claim 1 that is from a human cell line.

5. The cell according to claim 1 that is from a cell line from a tissue selected from the group consisting of kidney, brain, lung, heart, skin, liver, ovary, placental and tumour.

6. The cell according to claim 5 wherein the cell line is hepatic.

7. The cell according to claim 1 wherein in the instance that the cells from the cultured cell line do not have an inherent electron donating cytochrome P450 reductase (CPR) capacity the adenoviral expression vector further includes a nucleic acid sequence encoding CPR, the CPR being positioned in tandem with the nucleic acid sequences encoding the one or more different cytochrome P450s and separated therefrom by a further self-processing cleavage sequence.

8. The cell according to claim 1 that expresses 2, 3, 4, 5, 6, 7, 8 or more metabolically active or functional P450s.

9. The cell according to claim 1 wherein the metabolically active P450 is human and is selected from the group consisting of CYP1 family (CYP1A1; CYP1A2; CYP1B1), CYP2 family (CYP2A6; CYP2A13; CYP2B6; CYP2C8; CYP2C9; CYP2C19; CYP2D6; CYP2E1; CYP2F1; CYP2J2; CYP2R1; CYP2S1; CYP2W1), CYP3 family (CYP3A4; CYP3A5; CYP3A7; CYP3A43), CYP4 family (CYP4A11; CYP4A22; CYP4B1; CYP4F2) and CYP>4families (CYP5A1, CYP8A1, CYP19A1, CYP21A2, CYP26A1).

10. The cell according to claim 1 wherein expression of each of the P450s is driven by a self-processing cleavage sequence.

11. The cell according to claim 1 wherein the adenovirus expression vector further includes at least one reporter sequence or transgene and an associated self-processing cleavage sequence.

12. The cell according to claim 11 wherein the reporter transgene is a biomarker of a cellular stress response associated with early stages of toxicity.

13. The cell according to claim 12 wherein the cellular stress response is selected from the group consisting of oxidative stress (haemoxygenase 1 promoter); antioxidant response (ARE); inflammation (NF-kB); cell cycle advance (AP-1); DNA damage (p53); apoptosis (p21/Waf1); hypoxia (HRE) and other cell stress responsive sequences (XRE, Hsp70, GRE).

14. The cell according to claim 11 wherein readout from the reporter transgenes is either luciferase or epitope-tagged β-hCG.

15. The cell according to claim 1 wherein the expression of the at least one P450 is driven by a self-processing cleavage sequence.

16. The cell according to claim 1 wherein the self-processing cleavage sequence is a 2A site, sequence or domain or a 2A-like site, sequence or domain.

17. The cell according to claim 16 wherein the 2A sequence is from a mammalian virus selected from the group consisting of foot and mouth disease virus (FMDV), cardiovirus encephalomyocarditis virus (EMCV), Theiler's murine encephalitis virus (TMEV), equine rhinitis A virus (ERAV), equine rhinitis B virus (ERAV) and porcine teschovirus-1 (PTV-1; formerly porcine enterovirus-1) or is from an insect virus selected from the group consisting of Thoseaasigna virus (TaV), infectious flacherie virus (IFV), Drosophila C virus (DCV), acute bee paralysis virus (ABPV) and cricket paralysis virus (CrPV).

18. A cell from a cultured cell line that expresses one or more metabolically active cytochrome P450s, the cell comprising an adenovirus expression vector and one or more nucleic acid sequences encoding cytochrome P450s selected from the CYP1, CYP2, CYP3, CYP4 and CYP>4families, each selected cytochrome P450 being positioned in tandem with interposed 2A self processing sequences separating them and wherein the cell has a CPR function that is either inherent to the cell line or is provided by a nucleic acid sequence encoding CPR and 2A self-processing sequence.

19. The cell according to claim 18 wherein the cell is a human hepatocyte and the expressed functional P450s are human P450s.

20. The cell according to claim 18 further including at least one reporter sequence or transgene and an associated self-processing cleavage sequence.

21. A method of producing the cell of claim 1 comprising stably transfecting a cell from a cultured cell line with an adenovirus expression vector that comprises nucleic acid sequences encoding one or more different cytochrome P450s, the nucleic acid sequences encoding the one or more different cytochrome P450s being positioned in tandem and separated from one another by self-processing cleavage sequences, the vector optionally further including a nucleic acid sequence encoding CPR, the CPR being positioned in tandem with the nucleic acid sequences encoding the one or more different cytochrome P450s and separated therefrom by further self-processing cleavage sequence.

22. The method according to claim 21 wherein the adenovirus expression vector further includes at least one reporter sequence or transgene and an associated self-processing cleavage sequence.

23. A method for modelling drug metabolism and/or screening candidate compounds for toxic effects via metabolic activation, comprising exposing the cell according to claim 1 to a drug or candidate compound.

24. A method of assessing human P450 metabolism of a candidate therapeutic in vitro in a cell, comprising exposing the cell according to claim 1 to the candidate therapeutic and measuring metabolite production.

25. A method of assessing potential toxicity of a candidate therapeutic in vitro as a result of human P450 metabolism of the candidate therapeutic, comprising exposing the cell according to claim 1 to the candidate therapeutic and measuring cytotoxic effects.


SEQUENCE LISTING (2 sequences)

SEQ ID NO: 1 - DNA, 72 bp, Artificial
  Modified FMDV 2A sequence
ctcgaggtgaaacagactttgaattttgaccttctcaagttggcgggagacgtggagtccaacccagggccc

SEQ ID NO: 2 - protein, 22 aa, Artificial
  Synthetic Construct
VKQTLNFDLLKLAGDVESNPGP